(19) United States
(12) Patent Application Publication
(10) Pub. No.: US 2022/0299311 A1
(43) Pub. Date: Sep. 22, 2022

(54) DETERMINATION OF A TUBE PRESSURE BY MEANS OF LASER INTERFEROMETRY AND APPARATUS HEREFOR

(71) Applicants: Fresenius Medical Care Deutschland GmbH, Bad Homburg (DE); FRESENIUS MEDICAL CARE AG & CO. KGAA, Bad Homburg (DE)

(72) Inventors: David HANNES, Frankfurt a. Main (DE); Robert LINDEMANN, Wiesbaden (DE); Jonas HELLHUND, Frankfurt a. Main (DE)

(73) Assignees: Fresenius Medical Care Deutschland GmbH, Bad Homburg (DE); FRESENIUS MEDICAL CARE AG & CO. KGAA, Bad Homburg (DE)

(21) Appl. No.: 17/636,592

(22) PCT Filed: Aug. 21, 2020
(86) PCT No.: PCT/EP2020/073513

§ 371 Date: Feb. 18, 2022

(30) Foreign Application Priority Data
Aug. 22, 2019 (DE) 10 2019 122 629.5
Sep. 3, 2019 (DE) 10 2019 123 527.8

(51) Int. Cl. G01B 9/02; G01L 11/02

(52) U.S. Cl. CPC G01B 9/02095; G01L 11/02

(57) ABSTRACT

The present invention relates to a method of observing a changing surface by means of laser interferometry, in particular by means of laser speckle interferometry, wherein the changing surface is preferably a surface of a tube and the method is used to determine the pressure in the tube. A further aspect of the invention relates to a corresponding apparatus.

The present invention relates to a method of determining a tube pressure by means of laser interferometry and to an apparatus herefor.

The determination of the tube pressure in a fluid-conducting tube is of relevance in a large number of areas of application. For example in extracorporeal blood treatment, in particular in dialysis, it is of great importance to know the pressure values in the blood system or in the extracorporeal blood circuit. The blood system here is preferably that part of a fluid-conducting system of an extracorporeal blood treatment machine (dialysis machine) that conducts blood from the patient to the dialysis machine and from there behind the dialyzer back to the patient again.

The pressure values are decisive for ensuring patient safety or the success of the treatment for various reasons. On the one hand, it has to be avoided that the blood is sucked in from the patient at too high a suction pressure (too great a vacuum) since otherwise a destruction of the blood components (hemolysis) can occur.

Too high an excess pressure (relative to the environmental pressure) also has to be avoided to prevent hemolysis. Different treatment parameters (e.g. the ultrafiltration rate, the transmembrane pressure, etc.) are furthermore dependent on the pressure in the blood tube. The pressure in the tube system is furthermore used as an indicator for errors. For example, pressure changes can indicate an unwanted disconnection, e.g. due to a slipping out of the needles from the patient, or a sucking of the needles to the vessel wall.

Substantially two methods have previously been used for pressure measurement with machines for extracorporeal blood treatment. The first solution takes place via a tube section in which an air column is specifically left in place. Changes in the pressure in the tube result in a direct and correlated change to the air column. This change is detected via a pressure measurement station.

The second solution takes place via a special plastic component (pressure dome) that is integrated in the tube system. It is here a plastic housing into which a membrane has been introduced. This membrane is read out via a pressure detector at the machine side.

The problems of the two solutions can be found in the special designs of the blood tube system. In the first solution, an additional piece of tube is required in which air is located. In addition, the contact of blood with air (air column) is problematic since blood clotting functions are activated here. A contact of blood and machine could additionally occur if the pressure increases so much that the air column is completely displaced from the tube.

The second solution requires an additional part that is complex in production, that has a high cost, and that has to be introduced into the tube system. There are additionally unfavorable application properties that can result in the destruction of the machine component.

It is the underlying object of the present invention to alleviate or remedy the problems known in the prior art. It is specifically an object of the present invention to provide a method of determining a pressure in the interior of the tube and to provide an associated apparatus by means of which method/apparatus the pressure in the interior of a tube (also called the tube pressure) can be reliably and accurately measured without patient safety suffering due to a contact of the fluid conducted in the tube with any measurement structures (air column, pressure dome, etc.). In other words, a contactless method of determining the pressure in the interior of a tube should be provided.

This object is achieved by a method in accordance with claim 1 and by an apparatus in accordance with claim 9. Further advantageous further developments of the invention are the subject of the dependent claims.

A first aspect of the present invention relates to a method of observing a changing surface by means of laser interferometry, in particular by means of laser speckle interferometry, wherein the changing surface is preferably a surface of a tube and the method is used to determine the pressure in the tube.

This method exploits the fact that the surface of a flexible/elastic tube changes when the pressure in the tube changes.

For example, the tube stretches as the pressure in the tube increases, whereby the diameter of the tube increases and the surface of the tube is expanded. Such changes to the tube or to the tube surface can be detected by means of laser interferometry and the pressure present in the tube can be deduced from them.

In other words, the pressure in the tube can thus be measured without the integrity of the tube being violated or a contact to a fluid/medium (such as blood) guided in the tube having to be established. The measurement of the tube pressure thus takes place contactlessly.

The tube pressure can in principle be determined by two alternative procedures by means of a laser-assisted method in accordance with the invention, in particular by laser interferometer.

In accordance with a first procedure, a detection of the change of the diameter/circumference of the tube can take place by a distance measurement using laser interferometry.

The stretching of the tube is here preferably detected in that the change of the diameter of the tube is detected. A known laser-based distance measurement can be used for this purpose such that the expansion of the tube along the measurement axis, i.e. along the extent of a laser beam used for the measurement, is recognized via the change in the distance of the tube surface by a laser preferably fixedly mounted relative to the tube. A conclusion on the diameter can be drawn from the expansion measured in this manner. In an idealized case, the cross-section of the tube is circular, for example.

In other words, the change of the diameter of the tube can be determined by means of laser interferometry by means of a laser-based distance measurement, in particular by a time of flight measurement or by laser triangulation interferometry, from which the pressure in the tube can be deduced.

It has proven advantageous in this respect for the tube to be introduced into a fixture or to be clamped in a guide, whereby the tube is, however, fixedly/stationarily, but releasably, arranged relative to the laser.

It has furthermore proven advantageous for the expansion or the diameter of the tube to be measured on at least two mutually oppositely disposed, non-clamped sides of the tube so that an exact insertion of the tube into the guide is not necessary.

In other words, a particularly accurate determination of the diameter of the tube can take place by a measurement of the diameter of the tube at a plurality of tube sections that are not deformed by the fixture/guide.

As part of this procedure, known laser distance measurement can be used to determine the diameter of the tube, e.g.: time of flight measurement (measurement of the time elapsed between the emission of a light pulse/laser pulse and the reception of reflected light by means of a sensor), laser interferometry (the phase position of incoming and outgoing light is compared in this process), laser triangulation interferometry (laser light is transmitted along two separate paths in this process: the light is conducted directly to the sensor along one path. This path serves as a reference. The light is radiated onto an object, e.g. the tube, along a second path and is reflected by said object before impacting the sensor).

In accordance with a second procedure, the expansion of the tube or of the tube surface is determined by means of laser speckle interferometry. In this process, a laser radiates onto the surface of the tube, wherein the surface roughness or the structuring of the surface of tube results in an interference pattern (“laser speckle”) that is detected by an optical sensor (for example CCD, CMOS, camera) and is thereupon preferably evaluated by an evaluation unit.

A change or deformation or stretching of the surface of the tube can be recognized with reference to the detected speckle pattern. It is not necessary to know the dimensions of the tube in other directions for this purpose, but the change or deformation or stretching of the surface of the tube can rather already be determined solely with reference to the detected speckle pattern.

An evaluation unit preferably determines the expansion of the area of the surface of the tube from the change of the speckle pattern.

A preferably partially or fully automatic pattern recognition can be used to analyze the speckle pattern in principle in this process. Machine learning can, for example, also be used to analyze the speckle pattern,

The changing surface is preferably arranged in a stationary or fixed manner relative to a laser light source and/or to a laser receiver or sensor during a measurement process by means of a method in accordance with the invention.

In other words, in accordance with the invention the observed surface does not move-apart from the change due to the expansion-relative to the laser light source and/or to the laser receiver or sensor as is the case with other applications of laser speckle interferometry (for example in computer mice). In accordance with the invention, observed changes of the speckle pattern are thus due to changes of the surface per se and not to a relative movement of the surface with respect to the measurement apparatus.

Again in other words, a change, in particular a stretching, of the changing surface is determined with reference to an interference pattern and/or a speckle pattern by means of laser interferometry and the pressure in the tube can be deduced therefrom.

It is also conceivable that, alternatively or additionally, shearography methods and/or a phase shift technology are used.

It has furthermore been found to be advantageous in practice if an analysis takes place, preferably while taking account of an observed speckle pattern, by means of which observed changes of the surface due to an (unwanted) movement of the surface can be distinguished from observed changes of the surface due to a change/stretching of the surface.

For example, it can be determined by means of an image analysis whether changes of the speckle pattern arise due to an unwanted movement of the surface (for example due to a slipping of the tube in the fixture, with a translatory movement of the total tube producing a linear shift of the pattern) or due to a change of the surface (for example, cylindrical, star-shaped, or spherical changes of the speckle pattern in which the individual speckles/spots move away from one another indicate a stretching of the surface). Signals can in this manner be better delineated from measurement artefacts due to a slipping of the tube.

In addition, it can be advantageously be determined by means of an evaluation unit whether a measurement value of the pressure in the tube (or a corresponding measurement value of the diameter of the tube or a corresponding speckle pattern) falls within a tolerance range and an alarm can be triggered if the measurement value is not within the tolerance range.

In this respect, for example, a plausibility check can take place in which the measured pressure values are compared with the pressures typically occurring/to be expected e.g. as part of a specific operating mode of a blood treatment machine. The time duration during which a certain pressure is measured can equally be taken into account.

The measurement can generally be carried out continuously or intermittently at certain time intervals by means of a method in accordance with the invention.

The tube is preferably fixedly, but releasably, arranged in a fixture/guide before the carrying out of the method, whereby the measurement accuracy of the method is improved.

A further aspect of the invention relates to an apparatus for determining a tube pressure by means of laser interferometry, in particular by means of laser speckle interferometry, having a laser light source or a source of coherent light, an optical sensor for detecting the light transmitted by the laser light source or by the source of coherent light, and an evaluation unit that is adapted to evaluate the optical signals detected by the optical sensor.

The optical sensor can, for example, be a CCD camera, a CMOS sensor, or a camera.

The apparatus furthermore preferably has a guide/fixture in which a tube can be fixedly, but releasably, placed. The guide/fixture can here be associated construction-wise or also only functionally with the apparatus.

It has proven advantageous in practice for the guide to be formed as a tube chicane that preferably has a U-shaped guide groove for receiving a tube. This embodiment permits a particularly easy fixing of the tube.

The laser light source or the source of coherent light and the sensor system for detecting the transmitted light are preferably integrated or installed in the chicane.

For a particularly safe guidance of the tube in the chicane, the chicane is preferably configured such that it surrounds a tube received therein on at least two sides, preferably on three sides, or even at least sectionally on four sides.

To further improve the measurement accuracy, the laser light source can transmit laser light of at least two different colors, with the light of the different colors preferably being radiated to the same or substantially the same point. Green and red diode lasers are suitable in practice.

An apparatus in accordance with the invention is preferably fixedly or releasably arranged at a tube, in particular at a tube of an extracorporeal blood treatment machine, to determine the pressure in the tube.

A further aspect relates to a system composed of an apparatus in accordance with the invention and a tube, wherein the tube has a structure optimized for the measurement in at least one section at which the tube pressure is to be determined or in a section that interacts with the apparatus in accordance with the invention.

To improve the measurement accuracy, the section can in particular be produced with especially thin walls and/or from a material whose properties are relatively independent of temperature (so that, for example, a stretching of the surface of the section originates from a change of the pressure instead of the temperature).

Alternatively or additionally, the tube section can have a particularly rough surface structure to amplify a speckle pattern or can have a particularly smooth structure to improve the reflection properties.

In general, the tube section can also be provided with a preferably predefined pattern with reference to which changes of the surface are measurable.

Another aspect of the invention relates to an apparatus for extracorporeal blood treatment comprising at least one apparatus in accordance with the invention.

Further advantages, features, and effects of the present invention result from the following description of preferred embodiments with reference to the enclosed drawings. The same reference numerals designate the same components or similar components in the drawings. There are shown:

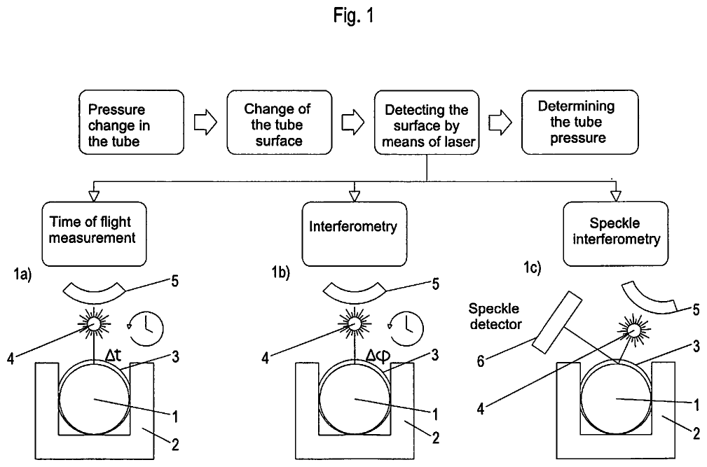

FIG. 1 an overview of different approaches in accordance with the invention for the contactless determination of the pressure in a tube;

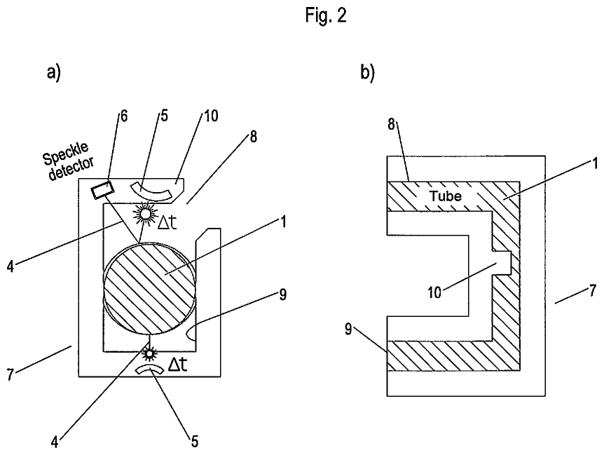

FIG. 2 a fixture/guide in the form of a chicane for a tube; FIG. 2a) shows a cross-section of the chicane with an inserted tube and FIG. 2b) shows a plan view of the chicane with an inserted tube; and

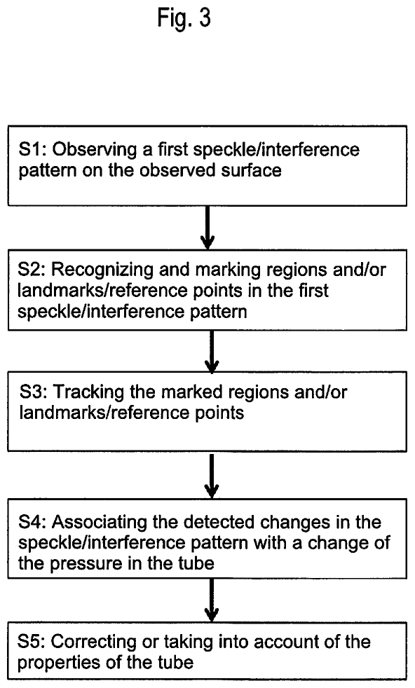

FIG. 3 a flow diagram of a method in accordance with the invention.

As shown in FIG. 1, the method in accordance with the invention is based on a changing surface (preferably a surface of a tube of an extracorporeal blood treatment machine, but the invention is not restricted thereto) being observed to draw conclusions on changes in the pressure in the tube with reference to changes of the surface.

Three variants are conceivable to detect/observe the changes of the surface by means of a laser and are shown schematically in FIG. 1:

It is assumed in all variants that a tube 1 is received in a fixture/guide 2. The diameter of the tube changes due to changes of the pressure in the tube 1, as is illustrated by the line 3 (in the example of FIG. 1, the pressure in the tube 1 increases and the diameter thus increases; the surface of the tube expands).

As shown in FIG. 1a), the measurement of the tube diameter can take place by means of a time of flight measurement. In this respect, laser light 4 is transmitted pulse-wise by a laser 5 and the time or the time interval Δt is determined starting from a transmission of the laser light 4 until an optical detector receives a reflection of the light.

A conclusion can be drawn on the distance of the surface from the laser 5 from the time of flight and the speed of light. Since the laser 5 is fixedly installed, any changes to the time of flight are due to changes of the distance between the surface of the tube 1 and the laser 5 and thus to changes of the pressure in the tube 1.

FIG. 1b) illustrates the laser interferometry. The term interferometry here comprises interferometry using triangulation, i.e. laser light is irradiated at one location and runs on two different paths until it is merged at a second location. The light of the two different paths interferes. The one path is direct, i.e. a straight line from the laser 5 to a detector/sensor. The second path typically runs over a reflection, e.g. at a surface of the tube 1. The light from the laser 5 is e.g. reflected on the tube wall of the tube 1. Small differences of the path length Δφ can be observed as an interference pattern. The interference pattern changes accordingly by a stretching of the surface due to a pressure change in the tube.

FIG. 1c) illustrates the laser speckle interferometry. The basis for the interference of the laser light comprises the surface of the tube 1 having a certain structuring/roughness. Constructive or destructive interference takes place in dependence on the expansion of recesses/valleys on the tube surface. A spot pattern of light, the speckle pattern, thereby results. This characteristic pattern changes with a change of the surface of the tube such as with an expansion of the tube due to a pressure change.

The speckle pattern is detected by means of a speckle detector 6 and is preferably analyzed by means of an evaluation unit. The evaluation of the speckle pattern requires an image detection that goes beyond a light sensor. A 1 D sensor strip is preferably provided, but it can also be a 2D sensor field (or a multidimensional sensor field) or a camera. An image evaluation detects the speckle pattern and its change and thus determines the pressure in the tube.

FIG. 2 shows a fixture/guide 7 in the form of a chicane for a tube 1. FIG. 2a) shows a cross-section of the chicane 7 with an inserted tube 1. The chicane 7 surrounds the tube 1 on three or, sectionally, also on four sides and has a reception opening 8 by means of which the tube 1 can be inserted into a groove 9.

As shown in FIG. 2a), the laser 5 and the speckle detector/sensor 6 are integral components of the chicane 7 or are fixedly installed therein. As shown in FIG. 2a), the laser 5 and the speckle detector/sensor 6 are preferably embedded in a holding section 10 that holds the tube 1 in the groove 9 and secures it against an unwanted dropping out.

FIG. 2b) shows a plan view of the chicane 7 with an inserted tube 1. The holding section 10 can be recognized particularly well in this representation. The chicane 7 surrounds the tube 1 at four sides at the holding section 10.

The chicane 7 additionally has a U-shaped design that enables a particularly reliable fixing of the tube 1 in the groove 9.

The chicane 7 is preferably produced in one piece from plastic. A multi-piece design from another material is, however, likewise conceivable.

As shown in FIG. 3, a method in accordance with the invention of observing a changing surface by means of laser interferometry, in particular by means of laser speckle interferometry, can comprise the following steps:
Step 1 (S1): Observing a first speckle/interference pattern on the observed surface;
Step 2 (S2): Recognizing and marking regions and/or landmarks/reference points in the first speckle/interference pattern;
Step 3 (S3): Tracking the marked regions and/or landmarks/reference points in order thus to detect changes of the speckle/interference pattern and therefore of the changing surface. The relative position with respect to one another and/or the absolute position of the marked regions and/or landmarks/reference points can be taken into account. On a pressure change in the tube, for example, the marked regions can move away from one another or can likewise be linearly displaced on a displacement of the tube. Light/dark transitions can furthermore be detected or tracked in the first speckle/interference pattern;
Step 4 (S4): Associating the detected changes in the speckle/interference pattern with a change of the pressure in the tube, for example with an expansion of the tube. If a plurality of pressures are to be measured in the tube, a calibration step is preferably carried out between the measurements;
Step 5 (S5): Correcting or taking account of the properties of the tube such as the elasticity, the diameter, the reflectivity, the surface roughness, etc. Step 5 can take place as part of the association of the changes in the speckle/interference pattern with a change of the pressure in the tube from Step 4. The correction can comprise a plausibility check.

1. A method of observing a changing surface by means of laser interferometry, in particular by means of laser speckle interferometry, wherein the changing surface is preferably a surface of a tube and the method is used to determine the pressure in the tube.

2. A method in accordance with claim 1, characterized in that the changing surface is arranged in a stationary manner relative to a laser light source and/or a laser receiver or sensor during a measurement process.

3. A method in accordance with claim 1, characterized in that the stretching of the changing surface is determined with reference to an interference pattern and/or a speckle pattern by means of laser interferometry and the pressure in the tube can be deduced therefrom.

4. A method in accordance with claim 3, characterized in that an analysis takes place, in particular taking account of an observed speckle pattern, by means of which observed changes of the surface due to a movement of the surface can be distinguished from observed changes of the surface due to a stretching of the surface.

5. A method in accordance with claim 1, characterized in that the change of the diameter of the tube is determined by means of laser interferometry by means of a laser-based distance measurement, in particular by a time of flight measurement or by laser triangulation interferometry, from which the pressure in the tube can be deduced.

6. A method in accordance with claim 1, characterized in that
it is determined by means of an evaluation unit whether a measurement value of the pressure in the tube is within a tolerance range; and in that
an alarm is triggered if the measurement value is not within the tolerance range.

7. A method in accordance with claim 1, characterized in that the tube is fixedly, but releasably, arranged in a fixture before the carrying out of the method.

8. An apparatus for determining a pressure in the interior of a tube by means of laser interferometry, in particular by means of laser speckle interferometry, comprising:
a laser light source;
an optical sensor for detecting the light transmitted by the laser light source; and
an evaluation unit that is adapted to evaluate the optical signals detected by the optical sensor.

9. An apparatus in accordance with claim 8, furthermore comprising a guide in which a tube can be fixedly, but releasably, placed.

10. An apparatus in accordance with claim 9, characterized in that the guide is formed as a tube chicane that preferably has a U-shaped guide groove for receiving a tube.

11. An apparatus in accordance with claim 9, characterized in that the laser light source transmits laser light of at least two different colors.

12. An apparatus in accordance with claim 8, characterized in that the apparatus is arranged at a tube of an extracorporeal blood treatment machine to determine the pressure of the tube.

13. A system of an apparatus in accordance with claim 8 and of a tube, characterized in that the tube has a structure optimized for the measurement at least in a section at which the tube pressure is to be determined, is in particular thin-walled and/or produced from a material whose properties are relatively temperature independent and/or has a particularly rough surface structure for amplifying a speckle pattern or has a particularly smooth structure for improving the reflection properties.

14. An apparatus for extracorporeal blood treatment having at least one apparatus in accordance with claim 8.
